(19) United States
(12) Patent Application Publication
(10) Pub. No.: US 2013/0273655 A1
(43) Pub. Date: Oct. 17, 2013

(54) ENGRAFTMENT POTENTIAL OF EMBRYONIC STEM CELLS AND PLURIPOTENT STEM CELLS USING MICRO RNAs

(71) Applicant: Shannon Eaker, Knoxville, TN (US)

(72) Inventor: Shannon Eaker, Knoxville, TN (US)

(21) Appl. No.: 13/795,656

(22) Filed: Mar. 12, 2013

Related U.S. Application Data
(60) Provisional application No. 61/609,489, filed on Mar. 12, 2012.

(51) Int. Cl. C12N 15/113

(52) U.S. Cl. CPC C12N 15/113
USPC 435/375; 536/24.1

(57) ABSTRACT

Methods and compositions for enhancing the engraftment potential of stem cells.

### CLAIM FOR PRIORITY

This application claims priority to U.S. Provisional Patent Application No. 61/609,489, filed Mar. 12, 2012; the contents of which are incorporated by reference herein in their entirety.

### BACKGROUND OF THE INVENTION

MicroRNA has been shown to be a critical component of cellular functions, self-renewal, differentiation and apoptosis. Recent studies have shown that microRNA miR-150 is directly involved in megakaryocyte-erythrocyte progenitor differentiation (Lu, Guo et al. 2008). Through a similar gain- and loss-of-function assay, it was shown that miR-150 drives megakaryocytic differentiation of Hematopoietic Stem Cell, or HSC progenitors, and does not produce erythrocytes. Ectopic over-expression of miR-181 in mouse bone marrow cells led to an increase of B-lineage cells both in vitro and in vivo (Chen, Li et al. 2004). A similar finding was seen in miR-155, in which miR-155-deficient mice lacked functional lymphocytes and were severely immunodeficient overall (Turner and Vigorito 2008). The microRNA expression profiles between growth factor-mobilized HSC's and peripheral blood leukocytes (PBL's) has been studied, and identified miRNA's up-regulated in all HSC's (miR-126, miR-10a, miR-221 and miR-17-92 cluster) (Jin, Wang et al. 2008). Additionally, miR-373 and miR-520c were shown to promote tumor invasion and metastasis in cell migration assays in a non-metastatic breast cancer cell line (Huang, Gumireddy et al. 2008). Methods for microRNA expression, gain- and loss-of-function have been well published, and new microRNA sequences are being submitted daily (Min and Chen 2006). Increasing the amount of microRNA's miR-221 and miR-222 in hUCB CD34₊ cells impaired erythropoiesis and decreased engraftment potential in NOD-SCID mice (Felli, Fontana et al. 2005).

The ability to develop and isolate patient-derived pluripotent cells for tissue-specific differentiation and transplantation will be a tremendous step forward in using regenerative medicine to treat and/or cure a wide variety of disorders. Given that only one third of patients requiring transplants have a sufficiently matched related donor, the use of patient-derived cell sources (such as iPS cells) are ideal for cell therapies. For future therapeutic applications, research must focus on how to avoid immune rejection once transplantation occurs. By using induced pluripotent stem (iPS) cells from the patients' own tissue, this issue can be avoided. The present invention provides for increased transplantation success by enhancing the ability of HSC to engraft in the host's hematopoietic niche through increasing, or inhibiting specific microRNAs. Also, the present invention is not limited to the hematopoietic stem cell niche (iPS-derived HSCs, cord blood and bone marrow stem cells), and can be extended to a variety of other tissue stem cell niches (e.g., neuronal, pancreatic) to study cellular development and differentiation for future iPS-based cellular therapies.

### SUMMARY OF THE INVENTION

The present invention relates to compositions and methods that enhance the engraftment potential of stem cells. The compositions comprise at least one microRNA agent, such as a microRNA mimic, or a microRNA inhibitor. The compositions may comprise a mixture of at least one microRNA mimic and at least one microRNA inhibitor. The methods include transfecting various stem cells with at least one of these microRNA agents so as to cause either a gain of function, or a loss of function. Any stem cell may be used, such as embryonic stem cells, adult stem cells, induced pluripotent stem cells, hematopoietic stem cells, mesenchymal stem cells, or neuronal stem cells. A preferable stem cell is a hematopoietic stem cell.

### BRIEF DESCRIPTION OF THE DRAWINGS

FIG. 1A shows the forward scatter vs side scatter of untransfected CD34+ cells isolated from cord blood.

FIG. 1B shows the fluorescence, plotted on a FL1 vs FL2 plot, of untransfected CD34+ cells isolated from cord blood and selected by the area shown on FIG. 1A.

FIG. 2A shows the forward scatter vs side scatter of CD34+ cells reagent isolated from cord blood and subsequently transfected with the mimicDY547 and 1 μl of DharmaFECT transfection reagent.

FIG. 2B shows the fluorescence, plotted on a FL1 vs FL2 plot, of CD34+ cells isolated from cord blood, subsequently transfected with the mimicDY547 and 1 μl of DharmaFECT transfection reagent and selected by the area shown on FIG. 2A.

FIG. 3A shows the forward scatter vs side scatter of CD34+ cells reagent isolated from cord blood and subsequently transfected with the mimicDY547 and 1 μl of Nanofectamine transfection reagent.

FIG. 3B shows the fluorescence, plotted on a FL1 vs FL2 plot, of CD34+ cells isolated from cord blood, subsequently transfected with the mimicDY547 and 1 μl of Nanofectamine transfection reagent and selected by the area shown on FIG. 3A.

FIG. 4A shows the forward scatter vs side scatter of CD34+ cells reagent isolated from cord blood and subsequently transfected with the mimicDY547 and 3 μl of Nanofectamine transfection reagent.

FIG. 4B shows the fluorescence, plotted on a FL1 vs FL2 plot, of CD34+ cells isolated from cord blood, subsequently transfected with the mimicDY547 and 3 μl of Nanofectamine transfection reagent and selected by the area shown on FIG. 4A.

### DETAILED DESCRIPTION OF THE INVENTION

In general, stem cells are undifferentiated cells which can give rise to a succession of mature functional cells. For example, a hematopoietic stem cell may give rise to any of the different types of terminally differentiated blood cells. Embryonic stem (ES) cells are derived from the embryo and are pluripotent, thus possessing the capability of developing into any organ or tissue type or, at least potentially, into a complete embryo.

“Pluripotent” may mean that the cell has the ability to develop into any cell derived from the three main germ cell layers or an embryo itself. When injected into SCID mice, a successful ES cell line will differentiate into cells derived from all three embryonic germ layers including: bone, cartilage, smooth muscle, striated muscle, and hematopoietic cells (mesoderm); liver, primitive gut and respiratory epithelium (endoderm); neurons, glial cells, hair follicles, and tooth buds (ectoderm).

Immortal cells are capable of continuous indefinite replication in vitro. Continued proliferation for longer than one year of culture is a sufficient evidence for immortality, as primary cell cultures without this property fail to continuously divide for this length of time (Freshney, Culture of Animal Cells. New York: Wiley-Liss, 1994). ES cells will continue to proliferate in vitro given specific culture conditions for longer than one year, and will maintain the developmental potential to contribute to all three embryonic germ layers.

Induced pluripotent stem cells (iPSCs) are adult cells that have been genetically reprogrammed to an ES cell-like state by being forced to express genes and factors important for maintaining the defining properties of ES cells, including expressing stem cell markers and being capable of generating cells characteristic of all three germ layers. Viruses are currently used to introduce the reprogramming factors into adult cells. Tissues derived from iPSCs will be a nearly identical match to the cell donor and thus probably avoid rejection by the immune system.

RNA silencing is a fundamental mechanism of gene regulation. Typically it uses double-stranded RNA (dsRNA) derived 21- to 28-nucleotide (nt) small RNAs to guide mRNA degradation, control mRNA translation or chromatin modification. Several hundred novel genes have been identified in plants and animals that encode transcripts that contain short dsRNA hairpins. Molecules referred to as microRNAs, are reported to be excised by dsRNA specific endonucleases from hairpin precursors. The molecule comprises a minimum number of ten moieties, preferably a minimum of thirteen, more preferably a minimum of fifteen, even more preferably a minimum of 18, and most preferably a minimum of 21 moieties. The molecule comprises a maximum number of fifty moieties, preferably a maximum of forty, more preferably a maximum of thirty, even more preferably a maximum of twenty-five, and most preferably a maximum of twenty-three moieties. A suitable range of minimum and maximum number of moieties may be obtained by combining any of the above minima with any of the above maxima.

Each moiety comprises a base bonded to a backbone unit. A base refers to any one of the nucleic acid bases present in DNA or RNA. The base can be a purine or pyrimidine. Examples of purine bases include adenine (A) and guanine (G). Examples of pyrimidine bases include thymine (T), cytosine (C) and uracil (U). Each base of the moiety forms a Watson-Crick base pair with a complementary base. Watson-Crick base pairs as used herein refers to the hydrogen bonding interaction between, for example, the following bases: adenine and thymine (A=T); adenine and uracil (A=U); and cytosine and guanine (C=G). The adenine can be replaced with 2,6-diaminopurine without compromising base-pairing. The backbone unit may be any molecular unit that is able stably to bind to a base and to form an oligomeric chain. Suitable backbone units are well known to those in the art.

For example, suitable backbone units include sugar-phosphate groups, such as the sugar-phosphate groups present in ribonucleotides, deoxyribonucleotides, phosphorothioate deoxyribose groups, N′3-N′5 phosphoroamidate deoxyribose groups, 2′O-alkyl-ribose phosphate groups, 2′-O-alkyl-alkoxy ribose phosphate groups, ribose phosphate group containing a methylene bridge, 2′-Fluororibose phosphate groups, morpholino phosphoroamidate groups, cyclohexene groups, tricyclo phosphate groups, and amino acid molecules.

The molecules of the invention comprise at least ten contiguous, preferably at least thirteen contiguous, more preferably at least fifteen contiguous, and even more preferably at least twenty contiguous bases that have the same sequence as a sequence of bases in any one of the molecules shown in Table 1. The molecules optimally comprise the entire sequence of any one of the molecules shown in Table 1.

For the contiguous bases mentioned above, up to thirty percent of the base pairs may be substituted by wobble base pairs. As used herein, wobble base pairs refers to either: i) substitution of a cytosine with a uracil, or 2) the substitution of an adenine with a guanine. These wobble base pairs are generally referred to as UG or GU wobbles. Further, up to ten percent, and preferably up to five percent of the contiguous bases can be additions, deletions, mismatches or combinations thereof. Additions refer to the insertion in the contiguous sequence of any moiety described above comprising any one of the bases described above. Deletions refer to the removal of any moiety present in the contiguous sequence. Mismatches refer to the substitution of one of the moieties comprising a base in the contiguous sequence with any of the above described moieties comprising a different base.

The additions, deletions or mismatches can occur anywhere in the contiguous sequence, for example, at either end of the contiguous sequence or within the contiguous sequence of the molecule. If the contiguous sequence is relatively short, such as from about ten to about 15 moieties in length, preferably the additions, deletions or mismatches occur at the end of the contiguous sequence. If the contiguous sequence is relatively long, such as a minimum of sixteen contiguous sequences, then the additions, deletions, or mismatches can occur anywhere in the contiguous sequence.

Furthermore, no more than fifty percent, and preferably no more than thirty percent, of the contiguous moieties contain deoxyribonucleotide backbone units. As stated above, the maximum length of the anti-microRNA molecule is 50 moieties. Any number of moieties having any base sequence can be added to the contiguous base sequence. The additional moieties can be added to the 5′ end, the 3′ end, or to both ends of the contiguous sequence.

MicroRNA molecules are derived from genomic loci and are produced from specific microRNA genes. Mature microRNA molecules are processed from precursor transcripts that form local hairpin structures. The hairpin structures are typically cleaved by an enzyme known as Dicer, which generates one microRNA duplex. See Bartel, Cell 116, 281-297 (2004) for a review on microRNA molecules. The article by Bartel is hereby incorporated by reference. Each strand of a microRNA is packaged in a microRNA ribonucleoprotein complex (microRNP). A microRNP in, for example, humans, also includes the proteins eIF2C2, the helicase Gemin3, and Gemin 4.

“Complement” or “complementary” as used herein may mean Watson-Crick or Hoogsteen base pairing between nucleotides or nucleotide analogs of nucleic acid molecules.

“Gene” used herein may be a genomic gene comprising transcriptional and/or translational regulatory sequences and/or a coding region and/or non-translated sequences (e.g., introns, 5′- and 3′-untranslated sequences). The coding region of a gene may be a nucleotide sequence coding for an amino acid sequence or a functional RNA, such as tRNA, rRNA, catalytic RNA, siRNA, miRNA and antisense RNA. A gene may also be an miRNA or cDNA corresponding to the coding regions (e.g., exons and miRNA) optionally comprising 5′- or 3′-untranslated sequences linked thereto. A gene may also be an amplified nucleic acid molecule produced in vitro comprising all or a part of the coding region and/or 5′- or 3′-untranslated sequences linked thereto.

“Host cell” used herein may be a naturally occurring cell or a transformed cell that contains a vector and supports the replication of the vector. Host cells may be cultured cells, explants, cells in vivo, and the like. Host cells may be prokaryotic cells such as E. coli, or eukaryotic cells such as yeast, insect, amphibian, or mammalian cells, such as CHO, HeLa.

“Identical” or “identity” as used herein in the context of two or more nucleic acids or polypeptide sequences, may mean that the sequences have a specified percentage of nucleotides or amino acids that are the same over a specified region. The percentage may be calculated by comparing optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces staggered end and the specified region of comparison includes only a single sequence, the residues of single sequence are included in the denominator but not the numerator of the calculation. When comparing DNA and RNA, thymine (T) and uracil (U) are considered equivalent. Identity may be performed manually or by using computer sequence algorithm such as BLAST or BLAST 2.0.

“Inhibit” as used herein may mean prevent, suppress, repress, reduce or eliminate.

“microRNA agent” as used herein may mean a molecule of microRNA as described above that can be a microRNA inhibitor, or a microRNA mimic. A “microRNA inhibitor” as used herein may mean a molecule of microRNA as described above that when introduced into a cell causes a loss of function of the gene to which the “microRNA inhibitor” is targeted. This loss of function can be a complete loss of function, or a reduced expression of the target gene. A “microRNA mimic” as used herein may mean a molecule of microRNA as described above that when introduced into a cell causes a gain of function of the gene to which the “microRNA mimic” is targeted. This gain of function can be a complete gain of function, or an increased expression of the target gene.

“Substantially complementary” used herein may mean that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50 or more nucleotides, or that the two sequences hybridize under stringent hybridization conditions.

“Substantially identical” used herein may mean that a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50 or more nucleotides or amino acids, or with respect to nucleic acids, if the first sequence is substantially complementary to the complement of the second sequence.

The microRNA may eventually become incorporated as single-stranded RNAs into a ribonucleoprotein complex known as the RNA-induced silencing complex (RISC). Various proteins can form the RISC, which can lead to variability binding site of the target gene, activity of microRNA (repress or activate).

The RISC may identify target nucleic acids based on high levels of complementarity between the microRNA and the target gene, especially by nucleotides 2-8 of the microRNA. Only one case has been reported in animals where the interaction between the microRNA and its target was along the entire length of the microRNA. This was shown for mir-196 and Hox B8 and it was further shown that mir-196 mediates the cleavage of the Hox B8 microRNA (Yekta et al 2004, Science 304-594). Otherwise, such interactions are known only in plants (Bartel & Bartel 2003, Plant Physiol 132-709).

A number of studies have looked at the base-pairing requirement between microRNA and its microRNA target for achieving efficient inhibition of translation (reviewed by Bartel 2004, Cell 116-281). In mammalian cells, the first 8 nucleotides of the microRNA may be important (Doench & Sharp 2004 GenesDev 2004-504). However, other parts of the microRNA may also participate in microRNA binding. Moreover, sufficient base pairing at the 3′ can compensate for insufficient pairing at the 5′ (Brennecke at al., 2005 PLoS 3-e85). Computation studies, analyzing microRNA binding on whole genomes have suggested a specific role for bases 2-7 at the 5′ of the microRNA in target binding but the role of the first nucleotide, found usually to be “A” was also recognized (Lewis et al., 2005 Cell 120-15). Similarly, nucleotides 1-7 or 2-8 were used to identify and validate targets by Krek et al., (2005, Nat Genet. 37-495).

The target sites in the microRNA may be in the 5′ UTR, the 3′ UTR or in the coding region. Interestingly, multiple microRNAs may regulate the same microRNA target by recognizing the same or multiple sites. The presence of multiple microRNA complementarity sites in most genetically identified targets may indicate that the cooperative action of multiple RISCs provides the most efficient translational inhibition.

MicroRNAs may direct the RISC to downregulate gene expression by either of two mechanisms: microRNA cleavage or translational repression. The microRNA may specify cleavage of the microRNA if the microRNA has a certain degree of complementarity to the microRNA target. When a microRNA guides cleavage, the cut may be between the nucleotides pairing to residues 10 and 11 of the microRNA. Alternatively, the microRNA may repress translation if the microRNA does not have the requisite degree of complementarity to the microRNA target. Translational repression may be more prevalent in animals since animals may have a lower degree of complementarity.

The microRNA agent of the invention may comprise a sequence of a microRNA, or a variant thereof. The microRNA sequence may comprise from 13-33, 18-24 or 21-23 nucleotides. The sequence of the microRNA may comprise a microRNA referred to in Table 1, or a variant thereof.

The present invention also relates to a method of using the microRNA agents of the invention to reduce expression of a target gene in a cell, tissue or organ, or a loss of function of a target gene. Expression of the target gene may be reduced by expressing a microRNA agent of the invention that comprises a sequence substantially complementary to one or more binding sites of the target microRNA. The microRNA agent may be a microRNA referenced in Table I or a variant thereof. The expressed microRNA may hybridize to a substantially complementary binding site on the target microRNA, which may lead to activation of RISC-mediated gene silencing. An example for a study employing over-expression of microRNA is Yekta et al., 2004, Science 304-594, which is incorporated herein by reference. One of ordinary skill in the art will recognize that the nucleic acids of the present invention may be used to inhibit expression of target genes using antisense methods well known in the art, as well as RNAi methods described in U.S. Pat. Nos. 6,506,559 and 6,573,099, which are incorporated by reference.

The present invention also relates to a method of using the microRNA agents of the invention to increase expression of a target gene in a cell, tissue or organ, or a gain of function of the target gene. Expression of the target gene may be increased by expressing a microRNA agent of the invention that comprises a sequence substantially identical to a microRNA or a variant thereof. Expression of the target gene may also be increased by expressing a microRNA agent of the invention that is substantially complementary to a portion of the binding site in the target gene, such that binding of the nucleic acid to the binding site may prevent microRNA binding.

The present invention also relates to a pharmaceutical composition comprising the microRNA agents of the invention and optionally a pharmaceutically acceptable carrier. The compositions may be used for diagnostic or therapeutic applications. The administration of the pharmaceutical composition may be carried out by known methods, wherein a nucleic acid is introduced into a desired target cell in vitro or in vivo. Commonly used gene transfer techniques include calcium phosphate, DEAE-dextran, electroporation, microinjection, viral methods and cationic liposomes.

### EXAMPLES

Experiments can be performed to determine specific microRNAs that increase the engraftment of HSC, either by increasing expression of particular microRNAs, or by loss-of-fucntion of particular microRNAs. Human iPS and CB cells can be obtained commercially (WiCell, Madison, Wis.; Stemgent, Cambridge, Mass.; Ontario Human iPS Cell Facility, Ontario, CA; BioCat GmbH, Heidelberg Germany; System Biosciences, Mountain View, Calif. for iPS, Cleveland Cord Blood Bank, Cleveland Ohio or Lonza, Basel, Switzerland for CB CD34+ cells), then characterized for full pluripotency and hematopoietic differentiation potential in vitro. The WiCell iPS cells are the original iPS cells from Jamie Thomson's 2007 study. Each cell type can be differentiated into hematopoietic stem cells/precursors. Side population (SP) analysis can be used to isolate the cells with increased engraftment potential and then microRNA gain-of-function/loss-of-function experiments can be conducted on the isolated cell types to elucidate each microRNA's role in cellular proliferation and their potential to induce hematopoietic differentiation in vitro. Malignant fibroblast cell lines (human patient- and disease-specific) can also be used in place of the iPS cells. Additionally, the cells that have been subjected to microRNA gain-of-function/loss-of-function experiments can be used to demonstrate the increased engraftment potential effect of the microRNAs. Table 1, below, outlines the microRNA's useful in the present invention.

TABLE I

| | |
|---|---|
| microRNA | Genbank Accession No. |

Gain-of-Function

| | |
|---|---|
| miR125a-5p | 406910 |
| miR-125b-1 | 406911 |
| miR-125b-2 | 406912 |
| miR-155 | 406947 |
| miR-130a | 406919 |
| miR196b | 442920 |
| miR-99a | 407055 |
| miR-126 | 406913 |
| miR-181c | 406957 |
| miR-193b | 574455 |
| miR-542-5p | 664617 |
| Let7e | 406887 |
| miR-10a | 406902 |
| miR-221 | 407006 |
| miR-222 | 407007 |
| miR-223 | 407008 |
| miR-17 | 406952 excel |
| miR-373 | 442918 |
| miR-520c | 574476 |
| miR-21 | 406991 |
| mIR-210 | 406992 |
| miR-129-1 | 406917 |
| miR-129-2 | 406918 |
| miR-520h | 574493 |
| miR-451 | 574411 |
| Let7b | 406884 |
| Let7c | 406885 |
| miR-100 | 406892 |
| miR-10b | 406903 |
| miR-142 | 406934 |
| miR-142-3p | 406934 |
| miR-142-5p | 406934 |
| miR-146a | 406938 |
| miR-146b | 574447 |
| miR-181a-1 | 406995 |
| miR-181a-2 | 406954 |
| miR-181b-1 | 406955 |
| miR-181b-2 | 406956 |
| miR-181d | 406957 |
| miR-30a-3- | 407029 |
| miR-99b | 407056 |
| miR-128-1 | 406915 |
| miR-128-2 | 406916 |
| miR-130b | 406920 |
| miR-16-1 | 406950 |
| miR-16-2 | 406951 |
| miR-24-1 | 407012 |
| miR-24-2 | 407013 |
| miR-29a | 407021 |
| miR-32 | 407036 |
| miR-421 | 693122 |
| miR-504 | 574507 |
| miR-103-1 | 100302238 |
| miR-103-2 | 100302238 |
| miR-107 | 406901 |
| miR-153 | 100314038 |
| miR-193a | 406968 |
| miR-302 | 407028 |
| miR-31 | 407035 |
| miR-383 | 494332 |
| miR-422b | 494334 |
| miR-425 | 494337 |
| miR-638 | 693223 |
| miR-663 | 724033 |
| Let-7a-1 | 406881 |
| Let-7a-2 | 406882 |
| Let-7a-3 | 406883 |
| miR-106a | 406899 |
| miR-109 | CUGGUCGAGUCGGCCUGCGC |
| miR-133 | 406922 |
| miR-191 | 406966 |
| miR-197 | 406974 |
| miR-200a | 406983 |
| miR204 | 406987 |
| miR-20a | 406982 |
| miR-213 | 406995 |
| miR-23a | 407010 |
| miR-23b | 407011 |
| miR-25 | 407014 |
| miR-26a | 407015 |
| miR-26b | 407017 |
| miR-27a | 407018 |
| miR-290 | 100422969 |
| miR-30b | 407030 |
| miR-30c | 407031 |
| miR-30d | 407033 |
| miR-30e-3p | 407034 |
| miR-335 | 442904 |
| miR-363 | 574031 |
| miR-9 | 407046 |
| miR-92-1 | 407048 |
| miR-92-2-p | 407049 |
| miR-93-1 | |
| miR-551b | 693135 |

Loss-of-Function

| | |
|---|---|
| miR-221 | 407006 |
| miR-222 | 407007 |
| miR-148a | 406940 |
| miR-145 | 406937 |
| miR-451 | 574411 |
| miR-520h | 574493 |
| miR-155 | 406947 |
| miR-223 | 407008 |
| miR-129-2 | 406918 |
| Let7b | 406884 |
| miR-142 | 406934 |
| miR-142-3p | 406934 |
| miR-142-5p | 406934 |
| miR-146a | 406938 |
| miR-146b | 574447 |
| mIR-150 | 406942 |
| miR-15a | 406948 |
| miR-15b | 406949 |
| miR-16-1 | 406950 |
| miR-16-2 | 406951 |
| miR-200c | 406985 |
| miR-203 | 406986 |
| miR-29a | 407021 |
| miR-33 | 407039 |
| miR-34a | 407040 |
| miR-153 | 100314038 |
| miR-18a | 406953 |
| miR-302 | 407028 |
| miR-31 | 407035 |
| miR-422b | 494334 |
| miR-425 | 494337 |
| miR-519c | 574466 |
| miR-143 | 406935 |
| miR-183 | 406959 |
| miR-29b | 407024 |
| miR-328 | 442901 |
| miR-342 | 442909 |
| miR-424 | 494336 |
| miR-503 | 574506 |

### Example I

### iPS Characterization and Embryoid Body (EB) Formation

iPS cells can be obtained commercially, and are tested for plurioptency by the manufacturer. iPS cells can be differentiated into embryoid bodies (EB's) as previously reported (Chadwick, Wang et al. 2003; Wang, Menendez et al. 2005; Wang, Cerdan et al. 2006). Ectodermal, endodermal and mesodermal germ layer formation within the EB's can be tested with antibodies against smooth muscle actin and cardiac troponin I (mesoderm), alpha fetoprotein (endoderm), and nestin (ectoderm) (Human Embryonic Germ Layer Characterization Kit, Millipore, Cat# SCR030). EB cells can be stained with the above antibodies, and visualized by flow cytometry or immunofluorescent microscopy.

EB single cell suspensions can be cultured on OP9 cells for 3 days. OP9 cells can be obtained from ATCC (American Type Culture Collection Cat# CRL-2749, Manassas, Va.). These are stromal bone marrow cells that were isolated from newborn op/op mouse calvaria, and have been previously shown to support hematopoietic growth prior to mouse transplantation (Wang, Menendez et al. 2005). The OP9 cells do not produce macrophage colony-stimulating factor (M-CSF) which inhibits embryonic stem cell differentiation into hematopoietic lineages. The cells can be cultured in Alpha Minimum Essential medium without ribonucleosides and deoxyribonucleosides, 2 mM L-glutamine, 1.5 g/L sodium bicarbonate, and 20% fetal bovine serum. As new iPS-to-hematopoietic differentiation protocols arise, they can also be tested for their HSC production efficiency (http://www.ncbi.nlm.nih.gov/pubmed/21037085, http://www.ncbi.nlm.nih.gov/pubmed/20948840).

### Example II

### Stem/Progenitor Cell Isolation Using the Side Population (SP) Assay

After 3 days on OP9 cells, stem/progenitor cell populations can be isolated and sorted by Side Population (SP) analysis as previously described (Eaker, Hawley et al. 2004). iPS/EB-derived suspension cells can be removed, then 200 U/mL of collagenase IV (Invitrogen) can be applied until adherent cells are removed by gentle pipetting. Total iPS/EB-derived cell suspension can be centrifuged, and resuspended at 1×10⁶ cells/ml in a total of 10 mls of Dulbecco's modified Eagle medium (DMEM, Invitrogen) with high glucose, 2% FBS (HyClone, Logan, Utah), and 10 mM N-(2-hydroxyethyl)piperazine-N′-(2-ethanesulfonic acid, HEPES) in a 15 ml conical tube. Hoechst 33342 (Sigma, St. Louis, Mo.) can be added at a final concentration of 5 μg/mL. The tube can be submersed in a water bath set at 37° C. for 90 minutes, with gentle inverting of the tube every 20 minutes to prevent cell clumping and settling. After 90 minutes, the cells can be centrifuged at 375 g for 6 minutes at 4° C. (in a pre-cooled rotor), and resuspended in cold PBS. It is critical to keep the cell suspension at 4° C. prior to and during sorting so that the cells retain the Hoechst dye that is taken up. Verapamil (Sigma) can be added to control populations at 50 μM to determine the desired population to be sorted. Samples can be analyzed on a FACSVantageSE/FACSDiVa (BD Biosciences) providing 30 mW of UV (351-356 nm) excitation. Observation of Hoechst 33342 fluorescence at its blue emission wavelength (with a 450/20 BP filter) and red emission wavelength (with a 675 LP filter) simultaneously on a Hoechst Blue (y-axis) versus Hoechst Red (x-axis) dot-plot can be used to identify the SP population. iPS/EB-derived SP⁺ cells can be sorted into the HSC media above, centrifuged and resuspended in the EB media with cytokines described above. The SP sorting can be performed on a FACSVantage SE/FACSDiVa (BD Biosciences) equipped with an Innova 302C krypton-ion laser (Coherent Inc., Santa Clara, Calif.) providing 30 mW of UV (351-356 nm) excitation. OP9 cells can also be stained as a control (previously found to be SP⁻). Alternatively, DyeCycle Violet (Invitrogen), a SP dye having similar emission patterns to Hoechst 33342, but excitable by violet laser lines (395-415 nm) can be used (Telford, Bradford et al. 2007).

### Example III—

### MicroRNA Gain-of-Function/Loss-of-Function on SP⁺ Stem/Progenitor Cells

Libraries of microRNA inhibitors and mimics are commercially available, and can be obtained from Dharmacon (miRIDIAN Mimic/Inhibitor Libraries, Dharmacon, Chicago, Ill.), along with DharmaFECT transfection reagent optimized for microRNA delivery. An alternative source of microRNA's can be obtained from Ambion (Foster City, Calif.). iPS-HSC-SP⁺GFP⁺ cells can be placed back onto OP9 cells for 2 days, then transferred to 96-well plates for microRNA mimic and inhibitor screening. iPS-HSC-SP⁺ cells can be tested for transfection cell density (starting with 5×10⁵ cells/ml in 100 μl/96-well). Transfection efficiencies can be monitored by fluorescently-labeled control microRNA's available from the manufacturer. It is also possible to test the expression regulation on a subset of microRNA mimics/inhibitors by Northern blotting. Alternatively, it is possible to use microRNA expression arrays on the Luminex multiplex platform. The Dharmacon transfection reagent has estimated transfection efficiencies for a variety of cell types (primary and transformed), ranging from 73-99% for microRNA mimics and inhibitors (a transfection efficiency of 93% was seen in the mouse ES cell line D3). The cells can be cultured at 37° C. in a humidified atmosphere containing 5% CO₂ overnight. 2 μM of the control microRNA along with 1.4 μl Dharmafect transfection reagent can be added to each well. After controls are concluded, microRNA mimics can be transfected into iPS-HSC-SP⁺GFP⁺ cells at 50 μM, and inhibitors at 25 μM, for 48 hours in iPS—HSC media.

### Example IV

### In Vitro Analysis of Stem/Progenitor Cells

Methylcellulose-based media can be used for the detection and quantification of human hematopoietic progenitors from iPS-HSC-SP⁺ cells. Cells will be plated in methylcellulose-based MethoCult GF H4434 medium at 4×10⁴ cells/ml (StemCell Technologies) containing FBS, BSA, 2-Mercaptoethanol, stem cell factor (SCF), GM-CSF, IL-3 and erythropoietin. Colonies can be analyzed 6-8 days later for total CFU counts for erythroid progenitors (CFU-E and BFU-E); granulocyte macrophage progenitors (CFU-GM, CFU-M, CFU-G) and multi-potential granulocyte erythroid, macrophage, and megakaryocyte progenitors (CFU-GEMM).

Further hematopoietic functional assays can be performed if needed, such as benzidine staining (red blood cells), phagocytosis analysis, superoxide anion production and acetylcholinesterase staining. Benzidine staining can be performed by adding 20 μl of benzidine staining solution (500 μl 0.2% benzidine in 3% acetic acid added to 50 μl H₂O₂ immediately prior to staining) to 2×10⁴ cells in 200 μl for 8 min. Phagocytosis of fluorescein-labeled E. coli K-12 BioParticles can be measured using the Vybrant Phagocytosis Assay Kit (Molecular Probes, Eugene, Oreg.) as described by the manufacturer. Cells (1.2×10⁵) can be suspended in 400 μl PBS supplemented with 2.5% mouse serum. Fluorescein-labeled BioParticles (100 μl) can be added to each sample and the samples incubated for 2 hours at 37° C. Prior to FACS analysis and fluorescence microscopy, 150 μl trypan blue can be added to each sample to quench extracellular fluorescence of non-phagocytosed particles. Superoxide anion production can be measured using the OxyBURST Green 2′,7′-dichlorodihydrofluorescein diacetate, succinimidyl ester reagent (H2DCFDA; Molecular Probes) as described by the manufacturer. Cells can be seeded into 96-well plates (1×10⁵/100 μA PBS per well) in the presence or absence of 100 ng/ml phorbol myristate acetate (PMA). OxyBURST Green H2DCFDA (50 μl) can be added to each well (5 μM final concentration) and the level of fluorescence (530 nm emission following 495 nm excitation) measured every 5 min up to 1 hour.

Acetylcholinesterase staining can be performed by a 6-hour incubation of cytospin preparations with acetylcholine substrate solution (0.5 mg/ml acetylcholine chloride, 75 mM dibasic sodium phosphate, 5 mM sodium citrate, 3 mM copper sulfate, 0.5 mM potassium ferricyanide; Sigma). Slides can be rinsed in distilled water and fixed in 95% ethanol for 10 min. Slides can be counterstained with May-Grunwald stain for 10 sec, washed in distilled water, allowed to air-dry and then mounted with Permount (Fisher Scientific, Pittsburgh, Pa.) for visualization of positive (brownish red) staining within the cytoplasm.

### Example V

### In vivo Transplantation of Stem/Progenitor Cells in Immunodeficient Mice

Four mouse strains (NSG, NOD-scid IL2Rγ^{null}, Rag2^{-/-}/γc^{-/-}, and B2 m^{-/-}/NOD/SCID) can be used for iPS—HSC-derived engraftment analysis. The intra-bone marrow injection method increases survival rates in injected mice, because i.v. administrations leads to donor cellular aggregation causing fatal emboli formation (Wang, Menendez et al. 2005). This study also showed a much lower total cell number required for successful engraftment compared to traditional i.v. studies (1.5×10⁵ instead of 1−2×10⁶). Female mice between 1-3 months in age can be irradiated with 3.25 Gy irradiation (¹³⁷Cesium source). Twenty-four hours later, the mouse can be anesthetized with Tribromoethanol/Avertin. A 25 g bottle of Tribromoethanol/Avertin in 15.5 mL amylalcohol can be prepared. In a sterile container, 1 mL of the concentrated solution can be mixed with 50 mL of 0.9% sterile saline for injection. The solution can be heated to 40° C. while maintaining sterility. A one-time dose of 0.15 mL/10 g can be injected by intraperitoneal injection once to each mouse. Published protocols for intra-bone marrow injection (Mazurier, Doedens et al. 2003; Wang, Kimura et al. 2003; Yahata, Ando et al. 2003) can then be used. The mouse can be immobilized, and 2×10⁵ cells can be injected in a total of 25 μl using a 27-gauge needle. The needle can be inserted into the joint surface of the tibia through the patellar tendon and then inserted into the BM cavity. All treated cell types can be injected in duplicate mice.

For human engraftment detection, the iPS—HSC's can be labeled using a lentiviral system along with green fluorescent protein (GFP) as a reporter. Lentiviral expression is not down-regulated (or silenced) during ES differentiation to the extent that non-lentiviral systems can be. The SINF-EF-GFP (self-inactivating (SIN) HIV-1-based lentivirus with elongation factor as a promoter) vector can be used with lentiviral 293T packaging cells (Ramezani, Hawley et al. 2000). This system has been shown to express GFP in mouse and human ES and ES-derived cells. Alternatively, other lentiviral vectors may be used. If, for some reason, silencing is seen during engraftment, antibodies against human cell surface markers can be used to identify the presence of human cells. Alternatively, the use of anti-human CD45 will be used as a marker (Hotta and Ellis 2008) (see below).

In addition to the use of SP⁺ cells for engraftment into immunodeficient mice, antibodies and immunophenotyping can be used to isolate cells for transplantation. PECAM-1⁺, Flk1⁺ and VE-cadherin⁺ (CD45⁻PFV⁺) cells can be isolated for transplantation as previously described (Wang, Menendez et al. 2005).

Every three weeks post-injection, mice can be monitored for GFP⁺ cells (having previously labeled the cells with lenti-GFP) in peripheral blood by retro-orbital eye bleeds. After 12 weeks post injection, the mice can be sacrificed by cervical dislocation. Mouse femurs can be removed, and bone marrow flushed using a 21 Gauge needle and syringe containing HSC media. The presence of human cells in both the injected femur, as well as the opposite non-injected contralateral femurs can then be determined. This allows for a better assessment of cellular homing, rather than only identifying the presence of human cells at the site of injection. Also, to differentiate between mouse and human cells, if necessary, the mouse anti-human nuclei antibody (Cat. No. MAB1281, Millipore) can be used. Lymphoid (CD45⁺/CD19⁺), myeloid (CD45⁺/CD33⁺) and erythroid (glycophorin Na⁺CD45⁺/human MHC-1⁺) hematopoietic lineages can be measured by flow cytometry on GFP⁺ cells. The following antibodies can also be used for human leukocyte detection after RBC lysis: CD45, CD3, CD4, CD8, CD14, CD19, CD20, CD56, CD133, BDCA-2, IgM, TCRαβ and TCRγσ (BD Biosciences). Bone marrow from GFP⁺ cells will be injected into secondary animals at 1×10⁵ cells/ml in 400 μl as above.

If no engraftment can be obtained using iPS cells, the hESC line H9 can be used. If any microRNA's are shown to increase engraftment, those microRNA results can be used on the iPS cell lines. Hematopoietic stem cells generated from the NIH-approved human embryonic stem cell line H9 can be injected as a positive control, in conditions previously published (Wang, Menendez et al. 2005).

### Example VI

### In Vitro Analysis of Stem/Progenitor Cells

Human-engrafted bone marrow stem cells can be isolated from engrafted mice, and plated for hematopoietic CFU/BFU secondary analysis as described above in Example IV.

### Example VII

### Optimizing Transfection Efficiency in CD34+ Cells

Transfection efficiency of iPS cells with the microRNAs can be first optimized in CD34+ cells isolated from cord blood using a transfection control microRNA mimic. Cord Blood CD34+ cells were prepared following the manufacturers protocol (Miltenyi Biotec Inc., Auburn, Calif.). Briefly, whole cord blood was filtered through a 30 μm nylon mesh (Millipore), washed in PBS, and resuspended in a final volume of 300 μL of PBS. Cell number was determined and the cell suspension was centrifuged at 300×g for 10 minutes. The supernatant was aspirated, and the cell pellet resuspended in 300 μL of PBS. 100 μL of FcR Blocking Reagent was added, and then 100 μL of CD34 MicroBeads added. The sample was mixed and incubated for 30 minutes in the refrigerator (2-8° C.). Fluorochrome-conjugated CD34 antibody (100 μg/ml) was added, and the sample incubated for 5 minutes in the dark in the refrigerator (2-8° C.). The cells were washed by adding 5-10 mL of PBS, then centrifuged at 300×g for 10 minutes. The supernatant was aspirated, and the cell pellet resuspended in 500 μL of PBS. The magnetic column was prepared by rinsing with 500 μL PBS. The cell suspension was applied onto the column, and the flow-through collected to contain unlabeled cells. The column was washed with 500 μL PBS, and the flow-through collected to contain unlabeled cells. The column was then removed from the magnetic separator, and 1 ml of PBS, and the magnetically-labeled cells flushed-out by firmly pushing the plunger into the column.

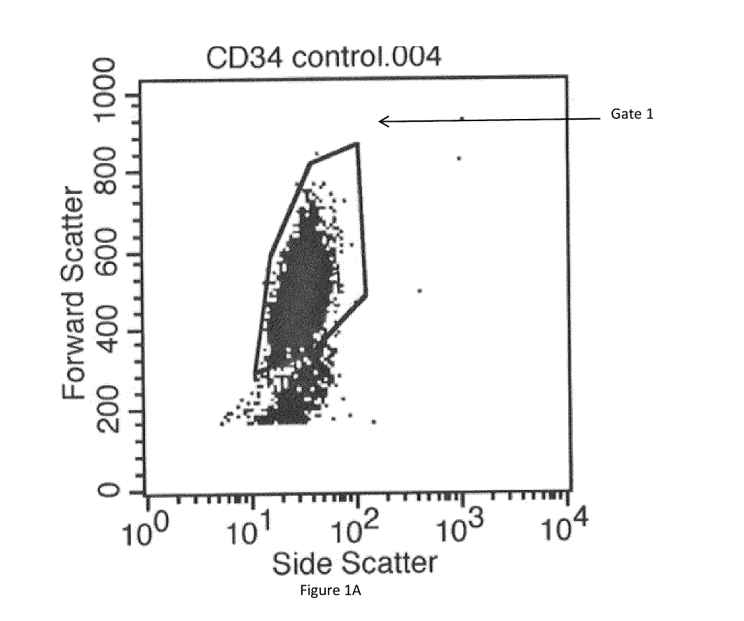

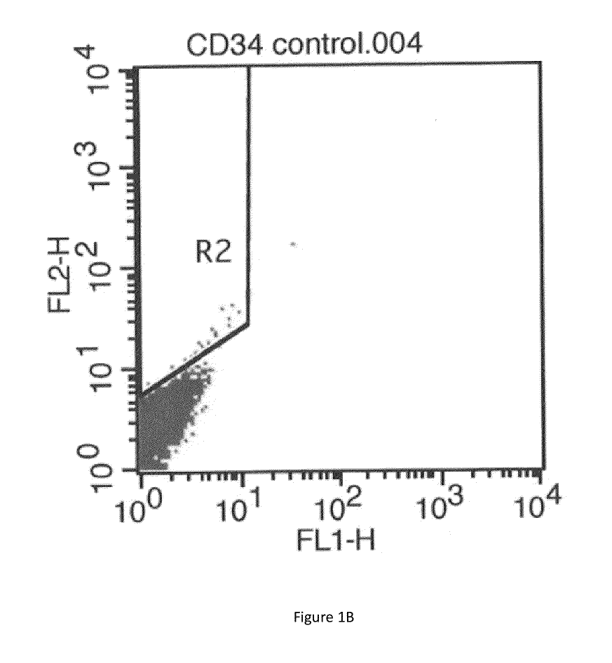

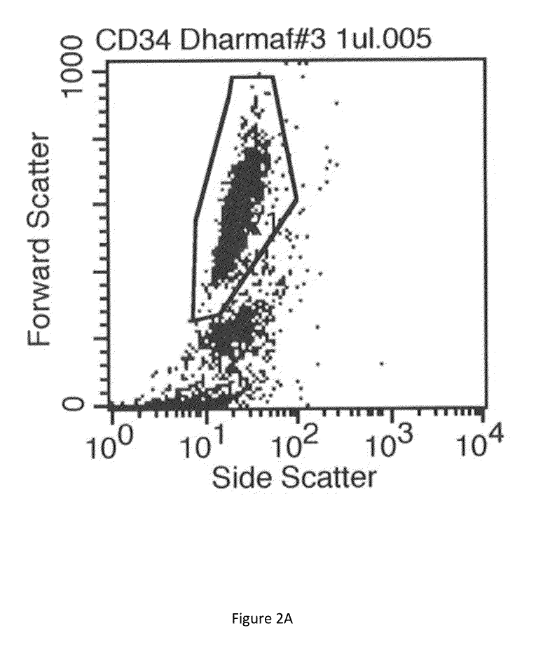

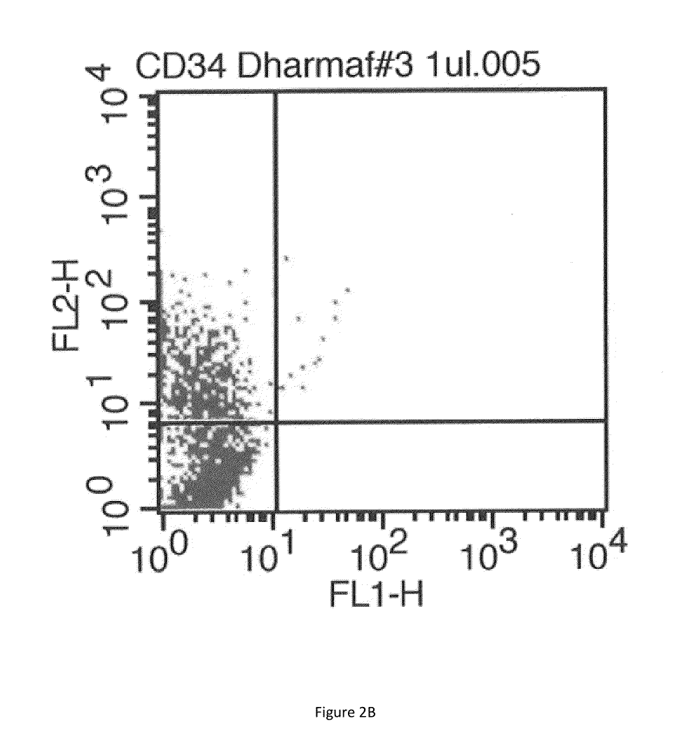

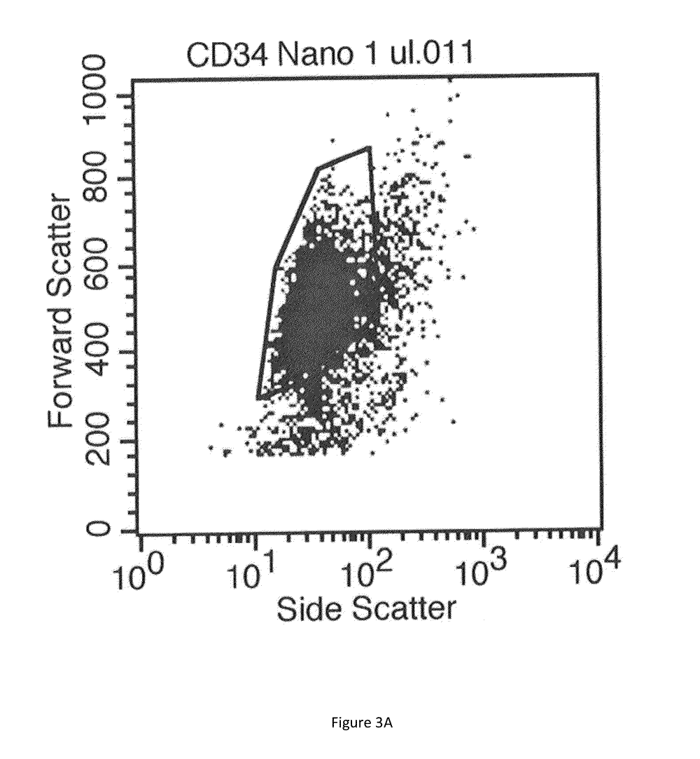

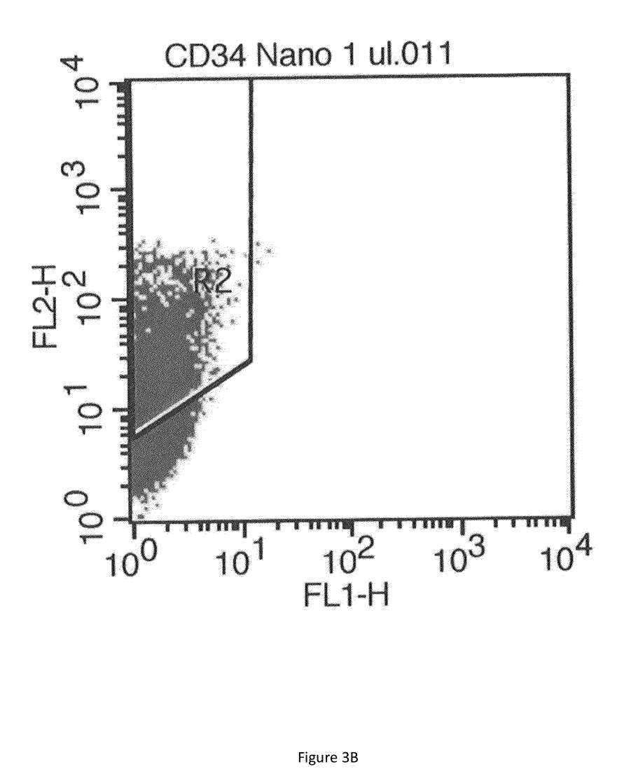

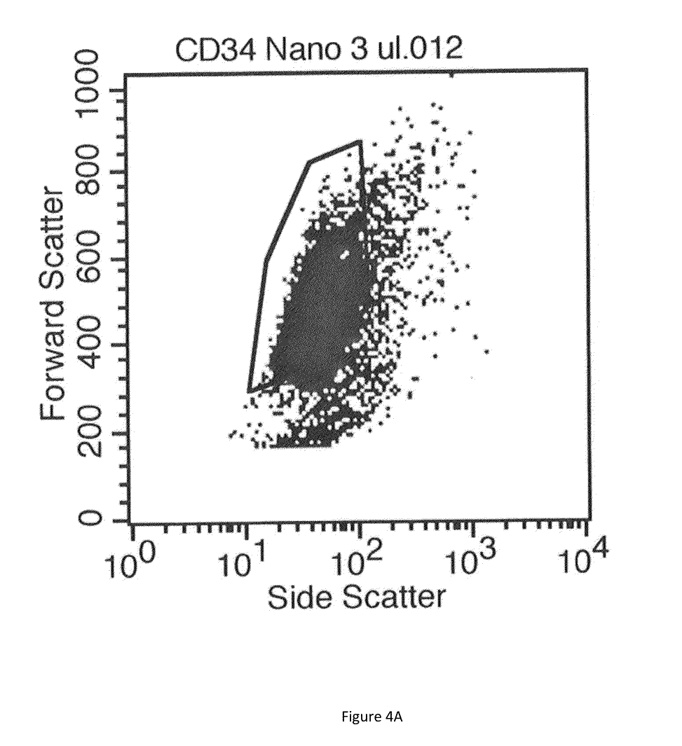

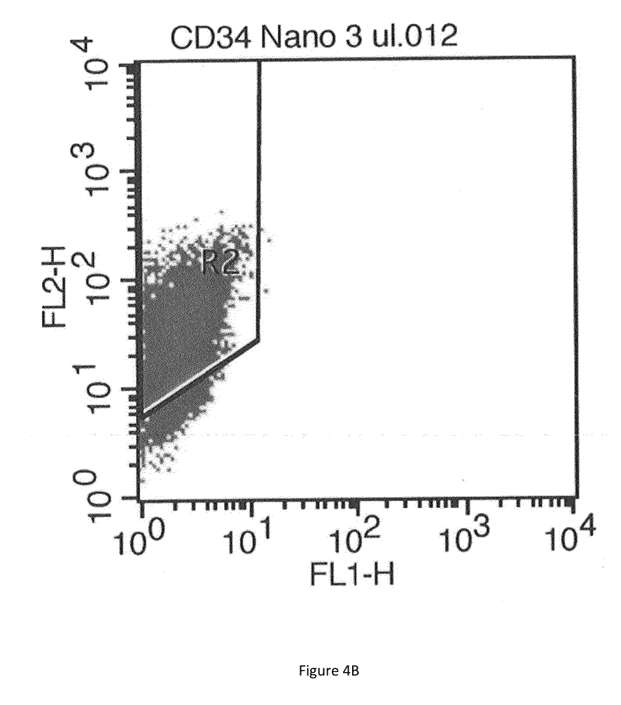

CD34+ cells isolated from cord blood as described above were counted and plated at 1×10⁵ per well in a 24-well plate with SCF, thrombopoietin (TPO), human Flt3 ligand (Flt3) (each at 50 ng/ml, PeproTech) in X-VIVO-10 serum-free media (Lonza, Walkersville, Md.) in a total volume of 250 μl. MimicDy547 (Thermo Fisher Scientific) was used as a control for monitoring transfections. The miRIDIAN microRNA Mimic Transfection Control is a Dy547-labeled microRNA mimic based on the C. elegans miRNA cel-miR-67 for monitoring delivery into mammalian cells. Delivery of MimicDy547 was measured by flow cytometry. Various commercial delivery/transfection reagents were tested for their ability to deliver MimicDy547 into human cord blood CD34+ cells. Reagents included Lipofectamine (Life Technologies), Nanofectamine (PAA Labs/GE Healthcare), and DharmaFECT (Thermo Fisher Scientific). Each transfection followed the manufacturers' protocol. The contents of both reagent preparations and cells were gently mixed individually by pipetting carefully up and down. Briefly, the tubes were incubated for 5 minutes at room temperature. The cells and reagents were mixed, and incubated for 20 minutes at room temperature. A volume of 150 μL of antibiotic-free complete medium was added to each mixture, and 250 μl of each transfection mixture was added to the designated well for a final volume of 500 μL transfection medium and a final MimicDy547 concentration of 25 nM. The cells were incubated at 37° C. in 5% CO2 for 24 hours. Subsequently, the cells were collected and analyzed for transfection efficiency by flow cytometry using a FACSCalibur (BD Biosciences). Briefly, the sample was visualized on a forward vs. side scatter plot and the relevant cell population was selected (e.g., Gate 1 on FIG. 1A). The fluorescence associated with the cells in the relevant population was then visualized on a FL2 vs FL1 plot (e.g., R2 on FIG. 1B) as the emission spectra of the Dy547 provides a maximum that is captured by the FL2 channel (550/570 nm). The results obtained using the various transfection agents are shown in FIGS. 2B, 3B and 4B and Table 2 below.

TABLE 2
Transfection Efficiency expressed as percentage of cells detected as
fluorescent following incubation with MimicDy547.

| Transfection Reagent | Transfection Efficiency |
|---|---|
| DharmaFECT | 38.9% |
| Nanofectamine (1 ml) | 54.7% |
| Nanofectamine (3 ml) | 76.3% |

What is claimed is:

1. A method of enhancing the engraftment potential of stem cells comprising:
transfecting said stem cells with at least one microRNA agent.

2. The method of claim 1, wherein the stem cells are selected from the group consisting of embryonic stem cells, adult stem cells, induced pluripotent stem cells, hematopoietic stem cells, mesenchymal stem cells, and neuronal stem cells.

3. The method of claim 2, wherein the stem cell is a hematopoietic stem cell.

4. The method of claim 1, wherein the at least one microRNA agent comprises at least one microRNA mimic that causes a gain of function.

5. The method of claim 4, wherein the at least one microRNA mimic is selected from the group consisting of miR125a-5p, miR-125b-1, miR-125b-2, miR-155, miR-130a, miR196b, miR-99a, miR-126, miR-181c, miR-193b, miR-542-5p, Let7e, miR-10a, miR-221, miR-222, miR-223, miR-17-92 cluster, miR-373, miR-520c, miR-21, mIR-210, miR-129-1, miR-129-2, and miR-520h.

6. The method of claim 1, wherein the at least one microRNA agent comprises a microRNA inhibitor that causes a loss of function.

7. The method of claim 6, wherein the at least one microRNA agent is selected from the group consisting of miR-221, miR-222, miR-148a, miR-145, miR-451, and miR-520h.

8. The method of claim 1, wherein the at least one microRNA agent comprises at least one microRNA mimic and at least one microRNA inhibitor.

9. The method of claim 8, wherein the at lest one microRNA mimic is selected from the group consisting of miR125a-5p, miR-125b-1, miR-125b-2, miR-155, miR-130a, miR196b, miR-99a, miR-126, miR-181c, miR-193b, miR-542-5p, Let7e, miR-10a, miR-221, miR-222, miR-223, miR-17-92 cluster, miR-373, miR-520c, miR-21, mIR-210, miR-129-1, miR-129-2, and miR-520h and the at least one microRNA inhibitor is selected from the group consisting of miR-221, miR-222, miR-148a, miR-145, miR-451, and miR-520h.

10. A composition for increasing the engraftment potential of stem cells comprising at least one microRNA agent.

11. The composition of claim 10, wherein the at least one microRNA agent comprises at least one microRNA mimic that causes a gain of function.

12. The composition of claim 11, wherein the at least one microRNA mimic is selected from the group consisting of miR125a-5p, miR-125b-1, miR-125b-2, miR-155, miR-130a, miR196b, miR-99a, miR-126, miR-181c, miR-193b, miR-542-5p, Let7e, miR-10a, miR-221, miR-222, miR-223, miR-17-92 cluster, miR-373, miR-520c, miR-21, mIR-210, miR-129-1, miR-129-2, and miR-520h.

13. The composition of claim 10, wherein the at least one microRNA agent comprises at least one microRNA inhibitor that causes a loss of function.

14. The composition of claim 13, wherein the at least one microRNA inhibitor is selected from the group consisting of miR-221, miR-222, miR-148a, miR-145, miR-451, and miR-520h.

15. The composition of claim 10, wherein the at least one microRNA agent comprises at least one microRNA mimic and at least one microRNA inhibitor.

16. The composition of claim 15, wherein the at lest one microRNA mimic is selected from the group consisting of miR125a-5p, miR-125b-1, miR-125b-2, miR-155, miR-130a, miR196b, miR-99a, miR-126, miR-181c, miR-193b, miR-542-5p, Let7e, miR-10a, miR-221, miR-222, miR-223, miR-17-92 cluster, miR-373, miR-520c, miR-21, mIR-210, miR-129-1, miR-129-2, and miR-520h and the at least one microRNA inhibitor is selected from the group consisting of miR-221, miR-222, miR-148a, miR-145, miR-451, and miR-520h.

17. The composition of claim 10, wherein said stem cells are selected from the group consisting of embryonic stem cells, adult stem cells, induced pluripotent stem cells, hematopoietic stem cells, mesenchymal stem cells, and neuronal stem cells.

18. The composition of claim 17, wherein said stem cells are hematopoietic stem cells.


SEQUENCE LISTING (1 sequence)

SEQ ID NO: 1 - RNA, 20 nt, Artificial Sequence
  Description of Artificial Sequence Synthetic oligonucleotide
cuggucgagucggccugcgc